(19) United States
(12) Patent Application Publication
(10) Pub. No.: US 2020/0031753 A1
(43) Pub. Date: Jan. 30, 2020

(54) CYCLOPROPYL ALKYL AMINES AND PROCESS FOR THEIR PREPARATION

(71) Applicant: Boehringer Ingelheim International GmbH, Ingelheim am Rhein (DE)

(72) Inventors: Zhengxu HAN, Shrewsbury, MA (US); Maurice A. MARSINI, Danbury, CT (US); Hao WU, New Milford, CT (US); Xingzhong ZENG, New Milford, CT (US)

(21) Appl. No.: 16/500,876

(22) PCT Filed: Mar. 28, 2018
(86) PCT No.: PCT/US2018/025072

§ 371 Date: Oct. 4, 2019

Related U.S. Application Data
(60) Provisional application No. 62/482,250, filed on Apr. 6, 2017.

(51) Int. Cl. C07C 51/41

(52) U.S. Cl. CPC C07C 51/412

(57) ABSTRACT

The present invention relates to a process of making a compound of formula (I). Wherein, R is as defined herein.

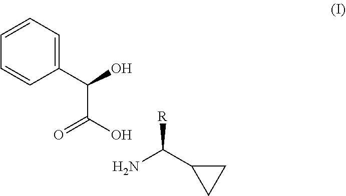

This application relates to a method of resolution of 1-cyclopropyl alkyl-1-amines which are building blocks in the preparation of substituted pyrazinones. These substituted pyrazinones can be used to prepare pharmaceutically active compounds containing a substituted pyrazinone ring system.

### BACKGROUND OF THE INVENTION

Cyclopropyl alkyl amines may be prepared by methods known in the literature and converted to substituted pyrazinones by adapting methods known in the literature. These substituted pyrazinone compounds can then be used to prepare pharmaceutically active compounds, such as ROR gamma modulators, containing a pyrazinone ring. These ROR gamma modulators are useful in treating a variety of diseases and disorders that are mediated through this pathway. The diseases that may be treated include but are limited to psoriasis and other inflammatory diseases. The preparation of ROR gamma modulators, containing a substituted pyrazinone ring, is disclosed in U.S. Pat. No. 9,242,989, issued Jan. 26, 2016, “Compounds as modulators of ROR gamma”.

### DESCRIPTION OF THE INVENTION

The present invention is directed to a process of making a compound of formula I

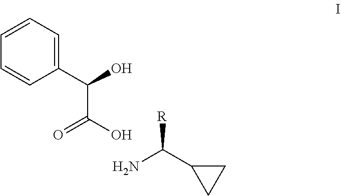

the process comprising: reacting an amine of formula II with a suitable base and mandelic acid, in a suitable solvent, to provide a compound of formula I:

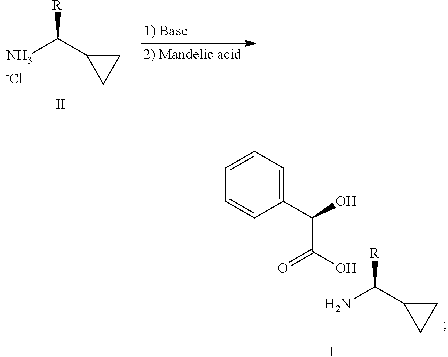

Wherein R in formula I and II, is a C₁₋₃ alkyl;

Non-limiting examples of bases useful in reaction step (1) include sodium hydroxide, potassium hydroxide, potassium t-butoxide, sodium t-butoxide, lithium t-butoxide, sodium hydride, potassium hydride, lithium hydride, sodium hexamethyldisilazide, potassium hexamethyldisilazide, lithium hexamethyldisilazide, sodium methoxide, potassium methoxide, lithium methoxide, sodium ethoxide, potassium ethoxide, lithium ethoxide, LDA, n-butyllithium, sec-butyllithium or t-butyllithium. Non-limiting examples of solvents useful for reaction step (1) include ethanol, methanol, propanol, water and mixtures thereof. Non-limiting examples of solvents useful for reaction step (2) include N,N-dimethylformamide, dichloromethane, ethyl acetate, hexane, heptane, acetonitrile, methyl tert-butyl ether, isopropyl acetate, toluene, and cyclopropylmethyl ether.

Starting amines of formula II may be prepared by methods known in the literature including (Lim et al., Discovery of 1-Amino-5H-pyrido[4,3-b]indol-4-carboxamide Inhibitors of Janus Kinase 2 (JAK2) for the Treatment of Myeloproliferative Disorders, J. Med. Chem., 2011, 54, 7334-7349), but not limited to the reaction sequence below:

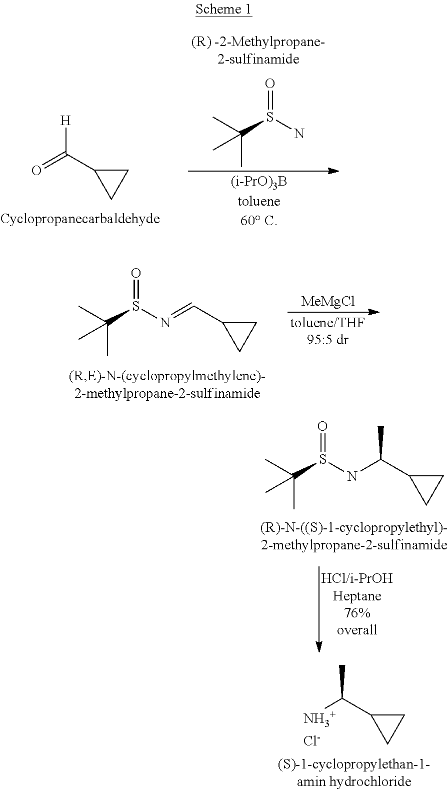

The mandelate salt of Formula (I) may be further transformed to Pyrazinone D, a key intermediate for the preparation of ROR gamma modulators, as shown below in Scheme 2 and disclosed in U.S. Pat. No. 9242989, issued Jan. 26, 2016, “Compounds as modulators of ROR gamma”.

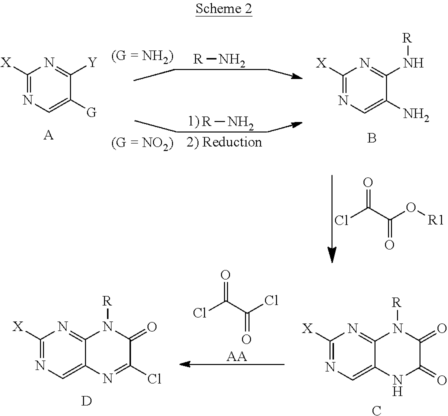

As illustrated in Scheme 2, a suitable pyrimidine of formula A, wherein G is NH₂, X is a suitable group for palladium-mediated cross coupling reactions (e.g., I, Br, Cl, or OSO₂CF₃), and Y is a suitable leaving group (e.g., Cl), may be reacted with a suitable amine or amine salt (e.g., hydrochloride salt) of formula R⁴NH₂ such as isopropyl amine in the presence of a suitable base (e.g., i-Pr₂EtN, or Et₃N) in a suitable solvent (e.g., n-butanol) and under a suitable reaction conditions such as an appropriate temperature (e.g., about 120° C.) to provide a compound of formula B. Alternatively, the said pyrimidine of formula A wherein G is a suitable synthetic precursor for NH₂ (e.g., a nitro group) may be reacted with a suitable amine or amine salt (e.g., hydrochloride salt) of formula R⁴NH₂ such as 1-methyl cyclopropylamine in the presence of a suitable reagent and solvent (e.g., i-Pr₂EtN and THF, respectively), and under a suitable reaction conditions such as an appropriate temperature (e.g., about −78° C. to about 25° C.) to afford an intermediate, which may be converted to a compound of formula B upon further reaction with suitable reagents (e.g., a NO₂ group that may be reduced with a suitable reagent such as SnCl₂). The selection of a suitable amine of formula R⁴NH₂ and pyrimidine of formula A for the aforementioned reaction by a person skilled in the art may be based on criteria such as steric and electronic nature of the amine and the pyrimidine. A diaminopyrimidine of formula B may be reacted with a suitable reagent such as chloro-oxo-acetic acid ethyl ester in a suitable solvent (e.g., acetone) and in the presence of a suitable base (e.g., K₂CO₃) to furnish a compound of formula C. A dicarbonyl compound of formula C′ may be reacted with a suitable dehydrochlorinating reagent such as oxalyl chloride in the presence of a suitable additive (e.g., a catalytic amount of DMF) in a suitable solvent (e.g., CH₂Cl₂), and under a suitable reaction conditions such as an appropriate temperature (e.g., about ambient temperature) to provide a compound of formula D.

The invention relates to the use of any compounds described above containing one or more asymmetric carbon atoms including racemates and racemic mixtures, single enantiomers, diastereomeric mixtures and individual diastereomers. Isomers shall be defined as being enantiomers and diastereomers. All such isomeric forms of these compounds are expressly included in the present invention. Each stereogenic carbon may be in the R or S configuration, or a combination of configurations.

Some of the compounds of the invention can exist in more than one tautomeric form. The invention includes methods using all such tautomers.

All terms as used herein in this specification, unless otherwise stated, shall be understood in their ordinary meaning as known in the art. For example, “C₁₋₆ alkoxy” is a C₁₋₆ alkyl with a terminal oxygen, such as methoxy, ethoxy, propoxy, butoxy. All alkyl, alkenyl, and alkynyl groups shall be understood as being branched or unbranched where structurally possible and unless otherwise specified. Other more specific definitions are as follows:

The term “alkyl” refers to both branched and unbranched alkyl groups. It should be understood that any combination term using an “alk” or “alkyl” prefix refers to analogs according to the above definition of “alkyl”. For example, terms such as “alkoxy”, “alkythio” refer to alkyl groups linked to a second group via an oxygen or sulfur atom. “Alkanoyl” refers to an alkyl group linked to a carbonyl group (C═O).

In all alkyl groups or carbon chains, one or more carbon atoms can be optionally replaced by heteroatoms such as O, S, or N. It shall be understood that if N is not substituted then it is NH. It shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo. As used herein, “nitrogen” and “sulfur” include any oxidized form of nitrogen and sulfur and the quaternized form of any basic nitrogen. For example, for a —S—C₁₋₆ alkyl radical, unless otherwise specified, shall be understood to include —S(O)—C₁₋₆ alkyl and —S(O)₂—C₁₋₆ alkyl.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

General Synthetic Methods

The invention provides processes for making compounds of Formula (I). Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC) or LC-MS, if desired, and intermediates and products may be purified by chromatography on silica gel, recrystallization and/or preparative HPLC.

The example which follows is illustrative and, as recognized by one skilled in the art, particular reagents or conditions could be modified as needed for individual compounds without undue experimentation. Starting materials and intermediates used are either commercially available or easily prepared from commercially available materials by those skilled in the art.

### SYNTHETIC EXAMPLE

### Example 1

### Synthesis of (S)-1-Cyclopropylethan-1-amine (R)-(-)-mandelate

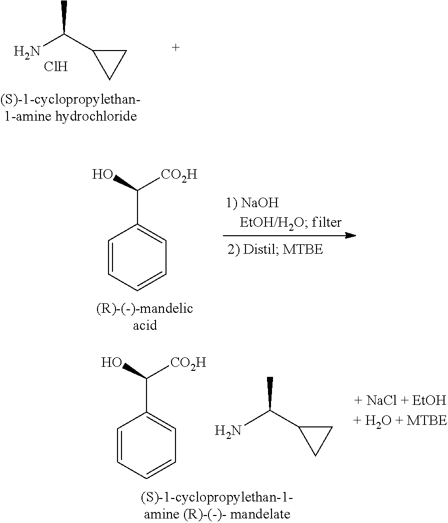

A solution of (S)-1-cyclopropylethan- 1-amine hydrochloride in ethanol (EtOH, 15 V) is treated with 1.0 eq of 25 wt % aqueous sodium hydroxide (NaOH) at 20-25° C. After 1 h at 20-25° C., the resulting slurry is filtered through Celite to remove sodium chloride (NaCl). To (R)-(-)-mandelic acid is added a solution of (S)-1-cyclopropylethan-1-amine hydrochloride) in ethanol, obtained above. EtOH/water is removed by azeotropic distillation to ˜4 V, after which methyl tert-butyl ether (MTBE, 12 V) is added at 60° C. After controlled cooling, the batch is filtered, and the wetcake is washed with 1:3 EtOH/MTBE. The product is obtained as a white solid in ˜83-87% isolated yield with a GC purity of >99 A %, >95¹H NMR wt %, and ˜99.5:0.5 er by GC analysis of the corresponding trifluoroacetamide derivative.

1. A compound having the following formula I: 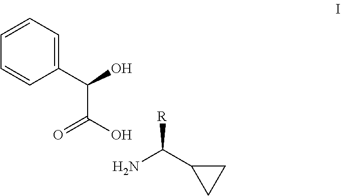
wherein R is a C₁₋₃ alkyl.

2. A process for making a compound of the formula I in claim 1, comprising reacting an amine of formula II with a suitable base and mandelic acid, in a suitable solvent, to provide a compound of formula I: 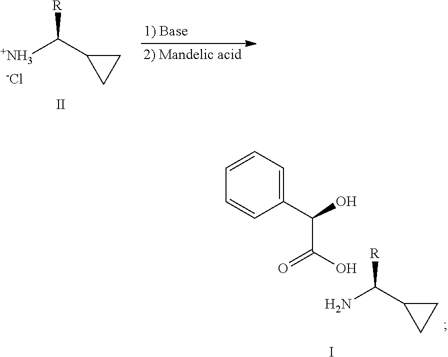
wherein R in formula I and II, is a C₁₋₃ alkyl.
